(19) United States
(12) Patent Application Publication
(10) Pub. No.: US 2014/0336304 A1
(43) Pub. Date: Nov. 13, 2014

(54) Durable dental material with improved transparent properties

(71) Applicant: HERAEUS KULZER GMBH, Hanau (DE)

(72) Inventors: Klaus RUPPERT, Maintal (DE); Alfred HOHMANN, Schmitten (DE)

(73) Assignee: HERAEUS KULZER GMBH, Hanau (DE)

(21) Appl. No.: 14/268,405

(22) Filed: May 2, 2014

(30) Foreign Application Priority Data
May 8, 2013 (DE) 10 2013 007 894.6

(51) Int. Cl. A61K 6/083; C08K 3/22; C08K 3/36

(52) U.S. Cl. CPC A61K 6/083; C08K 3/36; C08K 3/22
USPC 523/116; 106/287.14

(57) ABSTRACT

A dental material comprising (a) at least one curable monomer and/or polymer component, and (b) at least one filler component comprising agglomerated oxide particles having a matrix and a doping component, whereby the matrix contains silicon dioxide and the doping component comprises zirconium dioxide, whereby the agglomerated oxide particles are incorporated into the cured polymer matrix of the dental material and are abraded only in layers together with the polymer matrix of the dental material during an abrasion process and do not chip away as individual, intact particles. Moreover, the cured dental material possesses high transparency.

This application claims priority of German Patent Application No. 10 2013 007 894.6 filed May 8, 2013, the entire contents of which are incorporated herein by reference.

The invention discloses a dental material comprising (a) at least one curable monomer and/or polymer component, and (b) at least one filler component comprising agglomerated oxide particles having a matrix and a doping component, whereby the matrix contains silicon dioxide and the doping component comprises zirconium dioxide, whereby the agglomerated oxide particles are incorporated into the cured polymer matrix of the dental material and are abraded layer-by-layer together with the polymer matrix of the dental material during an abrasion process and do not chip away as individual intact particles. Moreover, the cured dental material possesses high transparency.

In order to meet high aesthetic requirements, dental composites, such as filling composites or veneer composites, need to possess high transparency. Said transparency is usually attained through optimal adaptation of the refractive indices of the fillers and polymer matrix. However, the limits on the selection of both fillers and monomers are rather narrow in this context due to various physical and chemical constraints.

Glass fillers typically have refractive indices between 1.50 and 1.54. Many suitable monomers for dental materials also have a refractive index in this range. However, composites, which contain just glass filler, have unfavorable handling properties. For this reason, it is customary to add rheology-modifiers, such as, e.g., pyrogenic silicic acids (“Aerosils”), to said preparations. Pyrogenic silicic acids have a refractive index of 1.46, though, and are thus quite distant from the optimal refractive index (>1.50) for dental materials. Adding said rheology-modifiers therefore worsens the transparency of the dental material made from them.

EP 1227 781 B1 discloses a dental material of a curable resin with silicon dioxide particles distributed in the resin and at least one heavy metal oxide, whereby the silicon dioxide particles have an average diameter of less than 200 nm and are present in an amount in excess of 40% by weight, based on the weight of the dental material. The heavy metal oxide is added as a separate sol. EP 1225867B1 discloses a dental material with non-heavy metal oxide particles having an average diameter of less than 300 nm and an acid-modified heavy metal oxide in a curable resin. EP1229886B1 discloses another filling material for dental materials. The filling material is an amorphous cluster of an index of crystallinity of less than 0.1, comprising a non-heavy metal oxide and an amorphous heavy metal oxide in particulate form having a diameter of 100 nm or less. EP0518454A2 discusses that a dental material with zirconium dioxide of a particle size above 1 μm has insufficient opacity.

The invention is based on the object to devise a rheology modifier, which does not worsen the transparency of the composite made from it, which is known to be the case with silicon dioxide. Moreover, sufficient opacity is required, such that a material is to be provided, which, as a rheology modifier, provides a balance of a desired transparency and a certain level of opacity. It is another object to devise a rheology modifier which, in addition, has a beneficial effect on the abrasion properties of the dental composite made from it. Beneficial shall be interpreted to mean that the depth profile generated upon abrasion is as small as possible, i.e. the smallest volume possible is abraded, that the roughness of the dental material after curing is completed is low and increases as little as possible due to abrasion and/or the reflectivity is changed as little as possible. Low roughness minimizes the affinity for plaque. The reflectivity should change not at all or only little for a long period of time even after exposure to heavy strain. Similarly, the gloss stability of the cured dental material, i.e. the difference in gloss level before and after an abrasion experiment, should be improved markedly.

According to the invention, the object is met through the use of a specific ZrO₂-doped silicic acid as rheology modifier in the filler component. The ZrO₂ content of the oxide mixture of the rheology modifier in the filler component is adjusted in this context in the range of approx. 5-25% by weight ZrO₂ in order to adjust the refractive index.

The afore-mentioned objects are met according to the invention through the use of at least one filler component comprising agglomerated primary particles having an average agglomerate grain size of approx. 1-10 μm. Said fillers are not chipped away from the polymer matrix as intact particles during abrasion, e.g. in particular they are not chipped away as agglomerates, but rather are abraded layer-by-layer. Accordingly, this does not lead to an increase in the surface roughness, reduced gloss and increased plaque affinity.

According to the invention, the objects are met, e.g., through the use of the products, ZirkonSil 520 and ZirkonSil 535. It is particularly advantageous for the agglomerated oxide particles comprising silicon dioxide and zirconium dioxide to be functionalized on the surface by means of an organo-functional silane that can be incorporated by polymerization. In this context, an organo-functional silane that can react with the organic monomers and polymers, preferably an olefinic alkoxysilane, such as methacryloxy-functionalized or vinyl-functional alkoxysilane, is preferred. Silanization can proceed in-situ during the production of the dental material or earlier in a separate step. In the latter case, the inventive agglomerated oxide particles (agglomerates) are surface-modified by an organo-functional silane in a separate step. The agglomerated oxide particles thus modified can then be added to the dental material as rheology modifiers in the filler component.

An object of the invention is a dental material, in particular a polymerizable/curable dental material, comprising (a) at least one curable monomer and/or polymer component, and (b) at least one filler component comprising agglomerated oxide particles, wherein the oxide particles comprising a matrix and a doping component, whereby the matrix contains silicon dioxide and the doping component comprises zirconium dioxide. The agglomerated oxide particles (agglomerates) are added as rheology modifiers in the filler component.

Furthermore, it is preferred according to the invention that the zirconium dioxide doping component is present in at least one domain, i.e. in at least one region of the oxide particles. Moreover, it can be preferred that the doping component is present in the silicon dioxide matrix in crystalline form, at least in part. Moreover, the doping component is embedded, at least in part, in the silicon dioxide matrix. Preferably, the doping component forms at least one crystalline domain within the primary particles.

The agglomerated oxide particles comprise essentially agglomerated primary particles, in particular having a matrix made of silicon dioxide. The oxide particles, in particular the primary particles, are zirconium dioxide-doped, preferably micro-crystalline zirconium dioxide-doped. Preferably, the doping component is present as at least one domain, in particular as at least one crystalline domain.

Also preferred is a dental material, in which the agglomerated oxide particles comprise agglomerates of silicon dioxide primary particles doped with zirconium dioxide, whereby the agglomerates have a particle size of 0.1 μm or more to 12 μm or less, in particular of 0.6 to 12 μm; preferably the average particle diameter of the agglomerates is 2.6 μm to 3.5 μm, approx. 2.6 μm or approx. 3.5 μm, measured by means of a volume distribution (volume-weighted) in an aerosol dispersion. The agglomerates contain primary particles made of oxide particles of silicon dioxide doped with zirconium dioxide.

The dental materials according to the invention are aesthetically particularly pleasing and appear particularly natural, since the refractive index of the agglomerated oxide particles is between 1.49 and 1.55. Therefore, particularly preferred agglomerated oxide particles have a refractive index of 1.49 and 1.55, in particular of 1.50 to 1.53, particularly preferably of 1.51 to 1.53, more preferably of 1.516 to 1.524.

According to one embodiment of the invention, preferred dental materials and filler components are agglomerated oxide particles of silicon dioxide-containing primary particles, whereby the primary particles contain zirconium dioxide domains.

According to one embodiment of the invention, particularly preferred dental materials and filler components are agglomerated oxide particles of silicon dioxide-containing primary particles, whereby the primary particles comprise micro-crystalline domains (synonymous to domain) of 4 to 7 nm. Preferably, the oxide particles comprise micro-crystalline zirconium dioxide-containing domains, preferably domains consisting of zirconium dioxide, particularly preferably micro-crystalline domains consisting of zirconium dioxide. Moreover, it is preferred for the agglomerated oxide particles of the primary particle to comprise a specific surface area of 5 to 10 m²/g.

According to another embodiment, it is preferred for the dental material or the filler component of agglomerated oxide particles comprising the matrix and the doping component to correspond to a mixture of metal dioxides selected from silicon dioxide and zirconium dioxide.

Moreover, it has been evident that the dental material or the filler component meets the objects of the invention, such as the transparency requirements, low abrasion and low roughness, particularly well, if the agglomerated oxide particles comprise 1 to 25% by weight zirconium dioxide, relative to the total composition, i.e. the doping component in the oxide particles can account for 1 to 25% by weight, relative to the total composition (100% by weight), of the oxide particle, preferably the zirconium dioxide content is 10 to 25% by weight, more preferably 10 to 15% by weight. Particularly preferred agglomerated oxide particles comprise 85 to 90% by weight silicon dioxide and 10 to 15% by weight zirconium dioxide, relative to their total composition, whereby it is further preferred for the primary particles of the agglomerated oxide particles to comprise micro-crystalline domains of 4 to 7 nm and it is advantageous for the crystallinity index to be 0.6 to 0.7—as determined by means of the method of Windisch et al. (WO 01/30306A), and for the agglomerated oxide particles to be present in a form that is surface-modified by at least one organo-functional silane that is reactive with at least one monomer and/or polymer component. Said agglomerated oxide particles treated according to the invention show excellent properties in abrasion measurements, with respect to the gloss level, excellent transparency and very good values in measurements of the reflection and roughness after a toothbrush test.

Also preferred are dental materials comprising at least one filler component or filler components comprising the agglomerated oxide particles, in which the agglomerated oxide particles are present at up to 80% by weight, in particular from 5 to 80% by weight, advantageously up to 50% by weight, up to 20% by weight, preferably from 10 to 30% by weight, relative to the total composition. Alternatively also preferred are 5 to 35% by weight, more preferably from 5 to 30% by weight, even more preferably 5 to 20% by weight, yet more preferably 10 to 25% by weight, 15 to 25% by weight, 20 to 80% by weight, particularly preferably 20 to 30% by weight, more particularly preferably 15 to 25% by weight, alternatively approx. 20% by weight with a variation range of plus/minus 2.5% by weight. Accordingly, a subject matter of the invention are dental materials and/or filler components comprising agglomerated oxide particles of a ZrO₂-doped silicic acid, in particular in a dental material at a concentration of up to 80% by weight, in particular up to 50% by weight, advantageously up to 20% by weight. Excellent results are obtained with a content of 10 to 25% by weight agglomerated oxide particles in the dental material. The clear advantage of doping versus coating or simply mixing with zirconium dioxide is reflected in the high transparency and the good reflectivity as well as the low roughness even after the toothbrush test. A silicic acid coated with zirconium dioxide, such as the product, JE340, shows markedly poorer results in terms of transparency, reflection and roughness after a toothbrush test.

Moreover, dental materials comprising filler components or filler components, which comprise agglomerated oxide particles at a molar ratio of silicon dioxide and zirconium dioxide of 1 to 9, in particular at a molar ratio of 1 to 8 to 1 to 6, are preferred.

Also preferred is a dental material having a filler component or a filler component, which comprise agglomerated oxide particles, which comprise primary particles made up of silicon dioxide and zirconium dioxide, whereby the agglomerates have a particle size d₅₀ of 0.5 to 12 μm, according to the invention of 0.5 to 10 μm, in particular of 1 μm or more to approx. 9 μm, in particular the d₅₀ is in the range of 1.5 to 5 μm, preferably the d₅₀ is in the range of 2 to 4 μm, particularly preferably in the range of 2.6 to 3.5 μm. Moreover, it is preferred for the agglomerated oxide particles of the filler component to have a grain size distribution d₉₀ of 12 μm or less and a mean particle size d₅₀ of approx. 2.4 to 3.0 μm.

Moreover, it is preferred for the agglomerated oxide particles, in particular the agglomerate, more preferably the agglomerated primary particles, to be surface-modified by organo-functional silanes such that advantageously essentially all primary particles are present incorporated into the cured dental material by polymerization, at least in part, in particular are covalently bound multiply to the surrounding polymer. Depending on the production method, the agglomerated oxide particles can just as well be present as aggregates. One advantage of the oxide particles used according to the invention is that they are not cleaved into the primary particles by high shear forces, e.g. during the production process. In particular and independent of each other, neither (a) the agglomerated oxide particles, (b) the agglomerated and aggregated oxide particles and/or nor (c) the surface-modified agglomerated oxide particles, in particular primary particles modified by conversion products of organo-functional silanes, are not cleaved into the primary particles by high shear forces that occur during the production of dental materials.

Said high shear forces occur, in particular, in the three-roller mill, centrifugal mixer, planetary mixer or dissolver, etc., which are customarily used during the production of dental materials, such as, e.g., composites. Accordingly, the agglomerated oxide particles according to the invention are advantageously also present as aggregated oxide particles, whereby the aggregation of the primary particles can be adjusted by means of oxidic bonds right during the production of the oxide particles and/or by the silanization.

In particular the cleavage may only occur through abrasion layer-by-lay in the cured dental material in long tern use by a patient.

Also a subject matter of the invention is a dental material or a filler component comprising agglomerated oxide particles having a surface modification, in particular a hydrophobic surface modification, advantageously through surface occupation by an organo-functional silane. Preferred silanes are organo-functional silanes that are reactive with the monomer and/or polymer component and the oxide particles, such as olefinic-functionalized alkoxysilanes, such as linear, branched and/or cyclic alkenyl-, (meth)acrylate-, in particular (meth)acrylatealkylene- or urethane-functionalized alkoxysilanes or the hydrolysis and/or condensation products thereof. The organo-functional group of the alkoxysilanes advantageously comprises 2 to 20 C atoms, in particular 2 to 10 C atoms, which may be interrupted or substituted by hetero atoms.

Preferred organo-functionalized alkoxysilanes or conversion products thereof, in particular hydrolysis and/or condensation products thereof, by means of which the agglomerated oxide particles, preferably the primary particles, are surface-modified comprise methacryloxyalkylenetrialkoxysilane, whereby the bi-functional alkylene group comprises 1 to 8 C atoms, 3-methacryloxytrimethoxysilane, 3-methacryloxytriethoxysilane, 3-methacryloxypropyltriethoxysilane, 3-methacryloxypropyltrimethoxysilane, alpha-methacryloxypropyltrimethoxysilane.

A subject matter of the invention is a dental material, in particular a curable dental material, containing: (a) at least one curable monomer and/or polymer component, (b) at least one filler component comprising (b.1) at least agglomerated oxide particles, in particular as rheology modifier, having a matrix and a doping component, whereby the matrix comprises silicon dioxide and the doping component comprises zirconium dioxide, in particular (b.1) accounts for up to 80% by weight, (b.2) glasses, in particular dental glasses, accounting for 30 to 65% by weight, preferably 30 to 60% by weight, and, optionally, (b.3) further rheology modifiers, which are frequently also listed as fillers and vice versa, such as silicic acid, silicon dioxide, pyrogenic silicic acid, in particular accounting for 0 to 5% by weight, preferably 0 to 1% by weight, and/or mixtures of at least two of the afore-mentioned filler components, and, optionally, (c) at least one initiator, in particular accounting for up to 1% by weight, advantageously from 0.0001 to 1% by weight, and (d), optionally, at least one pigment, in particular accounting for up to 1% by weight, preferably 0.0001 to 0.1% by weight, relative to the total composition.

Advantageously, the filler component comprises up to 80% by weight, particularly advantageously 50 to 80% by weight, preferably 55 to 80% by weight, more preferably from 60 to 80% by weight, even more preferably 65 to 80% by weight, particularly preferably 70 to 80% by weight of components b.1, b.2, b.3, relative to the total composition of the dental material, whereby the dental glasses account for 30 to 60% by weight, preferably 40 to 55% by weight, relative to the total composition. The total composition of the dental materials adds up to 100% by weight.

It is preferred, according to the invention, not to use common rheology modifiers, which are often also referred to as fillers, such as unmodified silicic acid, silicon dioxide and/or pyrogenic silicic acid. According to the invention, it is preferably sufficient to exclusively use as filler component the agglomerated oxide particles according to the invention having a silicon dioxide matrix and zirconium dioxide as doping components in the dental material as rheology modifier. Common rheology modifiers comprising unmodified silicic acid, silicon dioxide and/or pyrogenic silicic acid are used, at most, in small amounts of 0 to 2.8% by weight, preferably of 0.001 to 1.75% by weight. Accordingly, it is preferable to use a mixture of (b.1) and (b.2) or, optionally, a mixture of (b.1), (b.2) and (b.3) in the dental material, in particular with the content being up to 80% by weight relative to the dental composition, whereby components (b.1), (b.2) and/or (b.3) can be present in any conceivable compositions. Advantageously, components (b.1), (b.2) and (b.3) each are present silanized independently of each other.

Further objects of the invention include a dental material having a filler component comprising agglomerated oxide particles and said agglomerated oxide particles as such, in particular having a matrix and a doping component, whereby the matrix comprises silicon dioxide and the doping component comprises zirconium dioxide, comprising agglomerates of primary particles containing silicon dioxide and zirconium dioxide, whereby the primary particles have an average particle diameter of at least approx. 3 to 70 nm, in particular of 10 to 50 nm (nanometers). According to the invention, the doping component is present in the form of at least one domain in the silicon dioxide matrix. Mixtures of just silicon dioxide and zirconium dioxide or a silicon dioxide with an external zirconium dioxide coating do not possess the properties attained according to the invention, as was evidenced based on the examples. The ZrO₂-doped filler component preferably consists of agglomerated primary particles with a predominating particle diameter of approx. 10-50 nm (nanometers). Moreover, the crystallinity index of the agglomerated oxide particles according to the invention is 0.6 to 0.7, as determined in accordance with Windisch et al., WO01/30306, U.S. Pat. No. 7,030,049, EP 1229886B1.

The dental material according to the invention can be a filling composite, veneer composite, green compact of an artificial tooth, green compact of a veneer, green compact of an inlay, green compact of an implant, green compact of a carrier material for a local release of pharmaceutically active substances, green compact of a carrier material for a local antibiotics therapy or a green compact of a cutting block for the manufacture of dental prostheses according to the CAD/CAM technique or at least a part of the afore-mentioned dental materials. In this context, a green compact shall be understood to mean the pre-formed and uncured or incompletely cured dental material. The green compact of a veneer pre-formed from the dental material can subsequently be cured completely in order to be further processed by mechanical means according to need.

According to a further embodiment of the invention, another subject matter of the invention is a filler component comprising agglomerated oxide particles having a matrix and a doping component, whereby the matrix contains silicon dioxide and the doping component comprises zirconium dioxide, and whereby the agglomerated oxide particles are surface-modified by an organo-functional silane, in particular by the conversion product of the silane. Organo-functional silanes shall be understood to be the afore-mentioned silanes as well as their conversion products on the surface of the oxide particles. Preferably, the agglomerated oxide particles are surface-modified by conversion products of olefinic alkoxysilanes, in particular of 3-methacryloxypropyltrimethoxysilane and/or 3-methacryloxypropyltriethoxysilane.

According to the invention, the dental material can comprise a total filler content of (b) filler components of 20 to 98% by weight, in particular of 70 to 95% by weight, comprising (b.1) at 10 to 35% by weight, in particular 10 to 30% by weight, preferably 10 to 25% by weight, particularly preferably 15 to 25% by weight, in particular 10 to 15% by weight, 15 to 20 or, alternatively, 20 to 25% by weight agglomerated oxide particles, and, in particular, silanized oxide particles having a silicon dioxide matrix and zirconium dioxide as doping component having a crystallinity index of 0.6 to 0.7, and/or (b.2) at least one dental glass accounting for 0 to 75% by weight, in particular for 10 to 65% by weight, particularly preferably for 40 to 60% by weight, such as 45 to 50 or 50 to 65% by weight, whereby it is preferable to use a mixture of dental glasses made up of 50 to 90% coarse and 10 to 50% fine dental glasses, which have a size ratio, relative to the mean particle size (d₅₀ value), of coarse to fine of 1:4 to 1:30, and, optionally, (b.3) 0.5 to 10% by weight non-agglomerated nano-fillers of particle sizes of 1 to 50 nm.

It is preferred to select as (a) curable monomer and/or polymer components for the dental material one or more monomers from the monomer mixture (i), (ii), and (iii):

(i). at least one monomer from the group of bisglycidylacrylate, alkoxylated pentaerythritoltetraacrylate, TCD-di-HEMA or TCD-di-HEA, in particular accounting for 5 to 20% by weight, in particular 9 to 20% by weight, preferably for 10 to 20% by weight, more preferably 10 to 17% by weight, relative to the total composition, and
(ii). at least 5 to 20% multi-functional cross-linker UDMA (diurethanedimethacrylate), in particular 10 to 15% by weight, relative to the total composition, and
(iii). optionally residual TEDMA (trimethyleneglycoldimethacrylate) and/or further multi-functional cross-linkers, in particular accounting for 0 to 5% by weight, preferably 0.001 to 3% by weight or less, relative to the total composition, whereby (i), (ii) and (iii) account for a total of 5 to 35% by weight of the dental material, preferably for 15 to 35% by weight, particularly preferably for 20 to 35% by weight, more preferably for 20 to 25% by weight,
c) up to 1% initiator(s) and
(b.2) optionally, in the filler component, at least one further dental glass of a different particle size than the coarse and fine dental glasses, whereby the fraction of the total composition accounted for by (i) monomers is 9 or 10 to 17% by weight. Non-agglomerated nano-fillers are generally known and are described, e.g., in WO 0130305 A1 or in DE 196 17 931 A1 using SiO₂ as an example. They can preferably be selected from the group SiO₂, ZrO2, TiO₂, Al₂O₃ and mixtures of at least two of these substances. They can be dispersed in organic solvents—as described in DE 196 17 931 A1—or can be added to water or water-containing solvent mixtures.

Particularly well-suited as dental glasses are barium glass powders, preferably barium glass-aluminium-borosilicate glasses, and/or strontium glass powders. The mean particle size of the coarse dental glasses preferably is 5-10 [micro]m, in particular approx. 7 [micro]m, and the mean particle size of the fine dental glasses is 0.5 to 2 [micro]m, in particular 1 [micro]m. Further dental glasses that may optionally be present have mean grain sizes, e.g., of 2-5 or 10-50 μm [micro]m.

Accordingly, the filler component can comprise dental glasses having a total of three or more grain fractions. It can also contain further, conventional, fillers that are customary in the field of dentistry, such as quartz ceramics, glass ceramics or mixtures thereof. Moreover, the composites can contain fillers for attaining an increased radiopacity. The mean particle size of the radiopaque filler is preferably in the range of 100 to 300 nm, in particular 180 to 300 nm. Well-suited as radiopaque fillers are, e.g., the fluorides of rare earth metals, i.e. the trifluorides of elements 57 through 71, described in DE 35 02 594 A1. Ytterbium trifluoride is a filler that is used particularly preferably, in particular ytterbium trifluoride having a mean particle size of approx. 300 nm. The amount of radiopaque filler preferably is 10 to 50% by weight, particularly preferably 20 to 30% by weight, relative to the total filler content (b) of the dental material. According to the invention, it is preferable to use, aside from the agglomerated oxide particles having silicon dioxide matrix and zirconium dioxide as doping component, a customary filler, in particular silanized silicic acid made hydrophobic, at only a very small content of less than 5% by weight, preferably less than 2.5% by weight, more preferably less than 1.5% by weight.

The dental material preferably comprises the following monomers or polymers as curable monomer and/or polymer component:

Monomers that are common in the field of dentistry are conceivable as monomers: Examples include mono-functional monomers for radical polymerization such as mono(meth)acrylates, methyl-, ethyl-, butyl-, benzyl-, furfuryl- or phenyl(meth)-acrylate, poly-functional monomers such as poly-functional acrylates and/or methacrylates, e.g. bisphenol-A-di(meth)acrylate, bis-GMA (an addition product of methacrylic acid and bisphenol-A-diglycidylether), UDMA (urethanedimethacrylate), e.g. an addition product of 2-hydroxyethylmethacrylate and 2,2,4-hexamethylenediisocyanate), di-, tri- or tetraethyleneglycoldi(meth)acrylate, decanedioldi(meth)acrylate, dodecanedioldi(meth)acrylate, hexyldecanedioldi(meth)acrylate, trimethylol propanetri(meth)acrylate, pentaerythritoltetra(meth)acrylate as well as butanedioldi(meth)acrylate. Bis-GMA, TEDMA (triethyleneglycoldimethacrylate), UDMA (urethanedimethacrylate) in particular 1,6-bis(methacryloxy-2-ethoxycarbonylamino)-2,4,4-trimethylhexane (UDMA), TCD-di-HEMA (bis-(methacryloyloxymethyl)tricyclo-[5.2.1.0²′⁶]decane) and TCD-di-HEA (bis-(acryloyloxymethyl)tricyclo[5.2.1.0²′⁶]decane) are particularly preferred.

At least one monomer selected from the following or mixtures thereof can be used as preferred cross-linker monomer: 2,2-bis-4-(3-methacryloxy-2-hydroxypropyl)-phenylpropane) (Bis-GMA), i.e. the conversion product of glycidylmethacrylate and bisphenol-A (OH group-containing), and 7,7,9-trimethyl-4,13-dioxo-3,14-dioxa-5,12-diazahexadecane-1,16-diyldimethacrylate (UDMA), i.e. the urethanedimethacrylate from 2 mol 2-hydroxyethylmethacrylate (HEMA) and 1 mol 2,2,4-trimethylhexamethylenediisocyanate (urethane group-containing). Moreover, conversion products of glycidylmethacrylate and other bisphenols, such as, e.g., bisphenol-B(2,2′-bis-(4-hydroxyphenyl)-butane), bisphenol-F(2,2′-methylenediphenol) or 4,4′-dihydroxydiphenyl, as well as conversion products of 2 mol HEMA or 2-hydroxypropyl(meth)acrylate an, in particular 1 mol, known diisocyanates, such as, e.g., hexamethylenediisocyanate, m-xylylenediisocyanate or toluoylenediisocyanate, are well-suited as cross-linker monomers.

The following multi-functional cross-linkers are conceivable aside from TEDMA and UDMA: diethyleneglycol-di(meth)acrylate, decanedioldi(meth)acrylate, trimethylolpropanetri(meth)acrylate, pentaerythritoltetra(meth)acrylate as well as butanedioldi(meth)acrylate, 1,10-decanedioldi(meth)acrylate, 1,12-dodecanedioldi(meth)acrylate.

The dental material can preferably also comprise the following monomers and/or polymers as curable monomer and/or polymer component: one or more ethylenically unsaturated compound(s) with or without acid function. For example acrylic acid esters, methacrylic acid esters, hydroxy-functional acrylic acid esters, hydroxy-functional methacrylic acid esters and combinations thereof. As well as mono-, di- or poly-(meth)acrylates, i.e. acrylates and methacrylates, such as methyl(meth)acrylate, ethylacrylate, isopropylmethacrylate, n-hexylacrylate, stearylacrylate, allylacrylate, glyceroltriacrylate, ethyleneglycoldiacrylate, diethyleneglycoldiacrylate, triethyleneglycoldimethacrylate, 1,3-propanediol(meth)acrylate, trimethylolpropane-triacrylate, 1,2,4-butanetrioltrimethacrylate, 1,4-cyclohexanediol-diacrylate, pentaerythritol-tetra(meth)acrylate, sorbitolhexacrylates, tetrahydrofurfuryl(meth)-acrylate, bis[1-(2-acryloxy)]-p-ethoxyphenyldimethylmethane, bis[1-(3-acryloxy-2-hydroxy)]-p-propoxyphenyldimethylmethane, ethoxylated bisphenol A-di(meth)acrylate and trishydroxyethylisocyanurate-isocyanurate trimethacrylate, (meth)acrylamides (i.e. acrylamides and methacrylamides), such as (meth)acrylamide, methylene-bis-(meth)acrylamide and diacetone (meth)acrylamide; urethane(meth)acrylates; the bis-(meth)acrylates of polyethylene glycols (preferably having a molecular weight of 200-500), co-polymerizable mixtures of acrylated monomers, and vinyl compounds such as styrene, diallylphthalate, divinylsuccinate, divinyladipate and divinylphthalate. Other well-suited compounds for radical polymerization comprise siloxane-functional (meth)acrylates and fluoropolymer-functional (meth)acrylates or mixtures of two or more compounds for radical polymerization can be used according to need.

The polymerizable component can also comprise hydroxy groups and ethylenically unsaturated groups in a single molecule. Examples of such materials comprise hydroxyalkyl(meth)acrylates, such as 2-hydroxyethyl(meth)acrylate and 2-hydroxypropyl(meth)acrylate; glycerol-mono- or di-(meth)acrylate; trimethylolpropane mono- or di-(meth)acrylate; pentaerythritol mono-, di- and tri-(meth)acrylate; sorbitol mono-, di-, tri-, tetra- or penta-(meth)acrylate and 2,2-bis[4-(2-hydroxy-3-methacryloxypropoxy)phenyl]propane (BisGMA) or mixtures of ethylenically unsaturated compounds. The curable or polymerizable component can comprise PEGDMA (polyethyleneglycoldimethacrylate having a molecular weight of approx. 400), GDMA (glyceroldimethacrylate), TEGDMA (triethyleneglycoldimethacrylate) and/or NPGDMA (neopentylglycoldimethacrylate) as well as mixtures containing these.

For initiation of the polymerization, the composites contain a polymerization initiator, for example an initiator for radical polymerization. Depending on the type of initiator used, the mixtures can be cross-linked cold, by radiation, i.e. UV-cross-linked, or can be polymerizable through the supply of heat.

Known peroxides, such as dibenzoylperoxide, dilauroylperoxide, tert.-butylperoctoate or tert.-butylperbenzoate, but also alpha, alpha′-azo-bis(isobutyroethylester), benzpinacol and 2,2′-dimethylbenzpinacol, can be used as initiators for the temperature-induced polymerization.

Conceivable photoinitiators are, for example, benzoinalkylethers or -esters, benzilmonoketals, acylphosphinoxides or aliphatic and aromatic 1,2-diketocompounds, such as, for example, 2,2-diethoxyacetophenone, 9,10-phenanthrenequinone, diacetyl, furil, anisil, 4,4′-dichlorobenzil and 4,4′-dialkoxybenzil or camphorquinone. The photoinitiators are preferably used in conjunction with a reducing agent. Examples of reducing agents include amines such as aliphatic or aromatic tertiary amines, for example N,N-dimethyl-p-toluidine or triethanolamine, cyanoethylmethylaniline, triethylamine, N,N-dimethylaniline, N-methyldiphenylamine, N,N-dimethyl-sym.-xylidine, N,N-3,5-tetramethylaniline and 4-dimethylaminobenzoic acid ethylester or organic phosphites. Common photoinitiator systems include, e.g., camphorquinone plus ethyl-4-(N,N-dimethylamino)benzoate, 2-(ethylhexyl)-4-(N,N-dimethylamino)benzoate or N,N-dimethylaminoethylmethacrylate.

2,4,6-Trimethylbenzoyldiphenylphosphinoxide is particularly well-suited as initiator for the UV light-induced polymerization. UV photoinitiators can be used alone, in combination with an initiator for visible light, an initiator for cold curing and/or an initiator for temperature-induced curing.

Radicals-forming systems, e.g. benzoyl- and/or lauroylperoxide, are used in combination with amines such as N,N-dimethyl-sym.-xylidine or N,N-dimethyl-p-toluidine, as initiators for the cold polymerization. Dual-curing systems can be used as well, e.g. photoinitiators in combination with amines and peroxides. The initiators are preferably used in amounts from 0.01 to 1% by weight, relative to the total mass of the mixture.

It can be expedient in cold polymerization for the composite material to be subdivided into two components that are intended to be mixed for curing. It is also feasible to provide the material appropriately such that it can be cured both by VIS and/or UV light and by mixing of two components.

Also a subject matter of the invention is a method for producing a dental material according to the invention, and a dental material obtainable according to said method, in which (a) at least one curable monomer and/or polymer component, and (b) a filler component comprising (b.1) at least agglomerated oxide particles having a matrix and a doping component, whereby the matrix comprises silicon dioxide and the doping component comprises zirconium dioxide, in particular accounting for up to 80% by weight, and, optionally, (b.2) glasses, (b.3) rheology modifiers and/or mixtures of at least two of the afore-mentioned filler components and, optionally, (c) an initiator and, optionally, (d), at least one pigment are being mixed.

Subsequently, the dental material can advantageously be formed initially in a step (1) and optionally be polymerized in a further step (2) and optionally be lacquered and/or processed mechanically in a further step (3).

Dental materials that can be cured according to the invention obtainable, in particular, through polymerization, have a transparency of 55% or more, preferably of 58% or more, 58.5% or more, 59.5% or more, more preferably of 60% or more, particularly preferably of 62% or more (illumination: 8 min Palatray CU, both sides, HiLite Power 180 sec. both sides), a gloss level difference of 80 or less after toothbrush abrasion (polished surface up to 1000/2500/4000-grit polishing paper, diamond suspension yellow/red/white, toothbrush abrasion simulation Willytec/SD-Mechatronik, toothbrush Hager & Werken, Odol-med-3, 10,000 cycles, sawtooth profile, equivalent to approx. 3-6 months of brushing), in particular of 60 or less, preferably of 55 or less, particularly preferably of 50 or less, according to the invention of 45 or less, to 40 or less. Moreover, the cured dental materials according to the invention comprise one, more or all of the aforementioned parameters, each independently. Dental materials that are cured according to the invention and are obtainable, in particular, through polymerization advantageously have a roughness of 35 μm depth or less, in particular of 30 μm or less, particularly preferably of 25 μm depth or less, after poppy seed abrasion, as determined in the following according to the corresponding exemplary embodiments. In addition or alternatively, cured dental materials have a roughness having a volume of 0.4000 mm³ or less, preferably a volume of 0.3500 mm³ or less, preferably a volume of 0.3000 mm³ or less, particularly preferably a volume of 0.2500 mm³ or less, as determined after the poppy seed abrasion test, volume in the surface texture.

Dental materials that are cured according to the invention and are obtainable, in particular, through polymerization advantageously have a reflection of 1.5% or more, in particular of 2.0% or more, preferably of 2.5% or more, particularly preferably of 3% or more, more particularly preferably of 4.0% or more, even more preferably of 4.5% or more, determined, in particular, after a toothbrush abrasion. Cured dental materials that are preferred according to the invention comprise at least two and up to all of the afore-mentioned parameters.

The filler component according to the invention is advantageously produced in situ, in that the filler component comprising agglomerated oxide particles having a matrix and a doping component, whereby the matrix contains silicon dioxide and the doping component comprises zirconium dioxide, is surface-modified with an organo-functional silane, such as an ethylenically unsaturated organo-functional silane. A preferred filler component comprises agglomerated oxide particles surface-modified by conversion products of olefinic alkoxysilanes, in particular of 3-methacryloxytrimethoxysilane and/or 3-methacryloxytriethoxysilane, more preferred 3-methacryloxyalkylanetrimethoxysilane and/or 3-methacryloxyalkylanetriethoxy-silane 3-methacryloxypropyltrimethoxysilane and/or 3-methacryloxy-propyltriethoxy-silane.

According to a further subject matter, the invention discloses a cured dental material obtainable by polymerization of an afore-mentioned dental material or obtainable by mixing, optionally by forming and polymerizing, an afore-mentioned dental material.

Furthermore, a subject matter of the invention is a dental material obtainable by optional mixing and forming as well as through polymerization, whereby the agglomerated oxide particles, in particular agglomerated and aggregated oxide particles, are incorporated into the dental material's polymer matrix thus obtained and are removed layer-by-layer together with the polymer matrix of the dental material during an abrasion process, rather than chipping away as individual intact particles.

Moreover, a subject matter of the invention is a dental material, in particular a cured dental material, obtainable by polymerization, whereby the agglomerated and/or aggregated oxide particles, preferably aggregated oxide particles, are present covalently incorporated by polymerization into a polymer matrix of the dental material, preferably the oxide particles are present aggregated and/or covalently incorporated into the polymer matrix, and are removed layer-by-layer together with the polymer matrix of the dental material during an abrasion process, rather than chipping away as individual intact particles. Dental material obtainable through polymerization, whereby the agglomerated oxide particles are present aggregated in a polymer matrix of the dental material and are preferably incorporated covalently into the polymer matrix. Dental material obtainable through polymerization, whereby the agglomerated oxide particles are optionally present aggregated in a polymer matrix of the dental material and are incorporated covalently into the polymer matrix.

Preferably, the cured dental material is one of the dental products specified below or is used for the manufacture thereof, comprising: artificial tooth, veneer, inlay, carrier material for a local release of pharmaceutically active substances, carrier material for a local antibiotics therapy or a cutting block for the manufacture of dental prostheses, tooth, full denture, bridge, in particular a bridge of 2 to 9 units, or a cutting block for the manufacture of dental prostheses according to the CAD/CAM technique or at least a part of the afore-mentioned dental materials.

The dental material can be processed into dental products through curing, for example into tooth fillings, dental blanks, tooth crowns and bridges, dental prostheses, orthodontic devices and the like.

Dental materials also include, for example: adhesives (e.g. dental and/or orthodontic adhesives), cements (e.g. glass ionomer cements, resin-modified glass ionomer cements, and/or orthodontic cements), primers (e.g. orthodontic primers), repair compounds, reinforcement agents (e.g. a restorative filling material), linings, sealing compounds (e.g. orthodontic sealants) and coatings.

The dental material comprising curable monomer and/or polymer component can just as well be present in the form of a paste or a shapeable mass that is being cured in order to form a dental product. Dental products shall also comprise a restored denture or a part thereof. Pertinent examples include filling materials, artificial tooth, artificial teeth, inlays, onlays, veneers, full and partial crowns, bridges, implants, implant abutments, copings, anterior fillings, cavity inserts, base liners, lining material, sealants (coating for teeth), dental prostheses, bridge infrastructure and other bridge structures as well as parts thereof, or orthodontic apparatus and devices and prostheses (e.g. partial or full dental prostheses).

### BRIEF DESCRIPTION OF THE DRAWINGS

The invention is illustrated in more detail through the examples presented in the following, and the figures, though without limiting the scope of the invention. Briefly:

FIG. 1: shows the agglomerated oxide particles having a silicon dioxide matrix and zirconium dioxide doping component at a resolution of 1 μm,

FIG. 2: shows the agglomerated and preferably aggregated oxide particles covalently incorporated into the cross-linked dental material as abraded surface at a resolution of 1 μm,

FIG. 3: corresponds to FIG. 2 except for higher resolution, and

FIG. 4: shows the agglomerated oxide particles comprising the primary particles containing the silicon dioxide matrix and zirconium dioxide domains at a resolution of 200 nm.

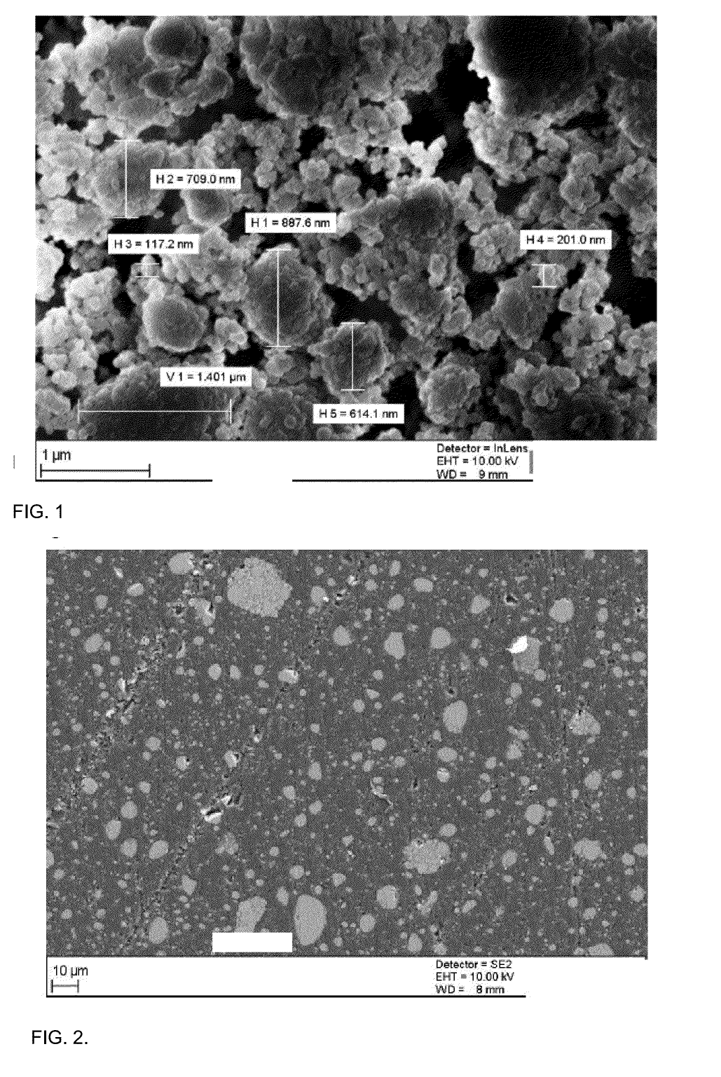

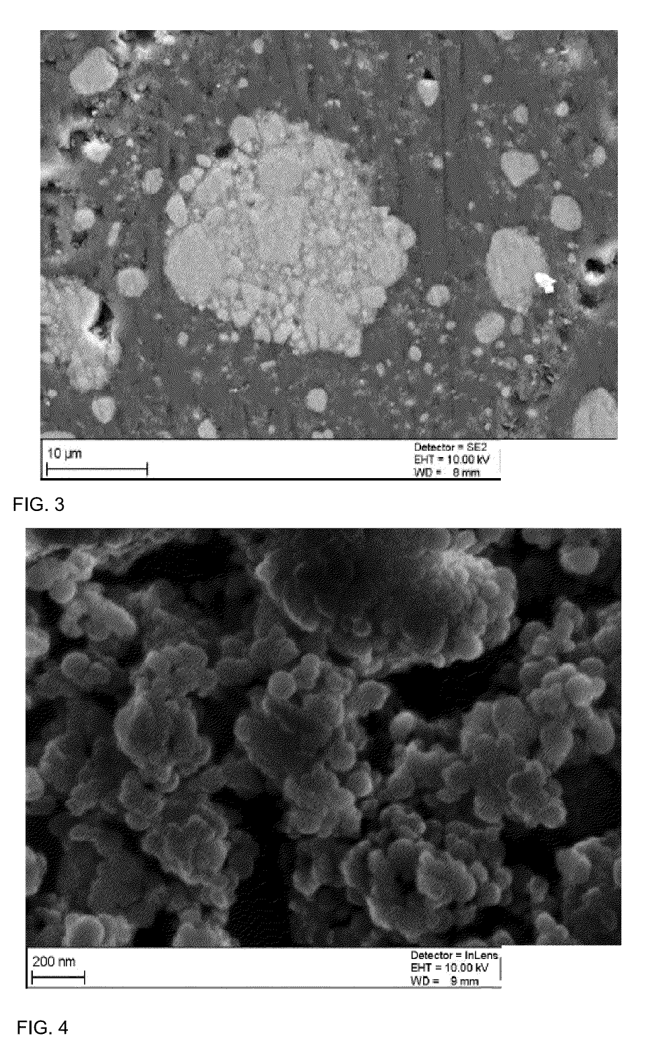

FIGS. 2 and 3 show SEM images of the ZrO₂-doped silicic acid filler incorporated into a methacrylate matrix, surface whetted and polished with polishing paper of decreasing grain size (up to 4,000 grit); in FIG. 3, the internal structure of the filler and the removal of the particle layer-by-layer by abrasion/polishing is clearly evident.

### Measurement of the Abrasion Properties

The abrasion properties of the dental materials were determined using test machines made by Willytec/SD-Mechatronik. In general, wear and tear of natural teeth or dental materials by in-vivo abrasion can occur due to a variety of mechanisms, such as through the antagonist tooth, wear due to abrasive particles in masticated food and/or wear due to cleaning with toothbrush and/or toothpaste. Said mechanisms of wear and tear can be simulated under laboratory conditions by various simulation methods.

3—Media abrasion (ACTA-method) for simulation of abrasion due to masticated food: The material to be tested is placed in a rotating wheel with 12 chambers and the wheel is run at 180 min⁻¹ in the opposite direction from an antagonist wheel made of steel (240 min⁻¹). Both wheels are supported as in a bearing in a suspension of water and poppy seed (poppy seed abrasion, MA) (110 g poppy:200 g water). The antagonist wheel is pressed against the test body wheel with a force of 20 N. Poppy seeds can move through the gap formed by the test body wheel and antagonist wheel and cause surface damage on the composite surface. Test machine 3-media abrasion/manufacturer: Willytec/SD-Mechatronik

Abrasion test: After 2×150,000 revolutions of the test body wheel (corresponds to a duration of use in the patient's mouth of approx. 3 years), the damage to the composite caused by poppy seeds is measured in each chamber and determined and analyzed in terms of depth and volume with a laser scanner. The detection of the depth profile is contact-less; the volume in mm³ and the mean depth in μm (micrometers) are measured.

The purpose of toothbrush abrasion is to simulate the abrasion that occurs by brushing with a toothbrush and toothpaste: The toothbrushes (a total of 8 per package) are pressed against the test body surface with a force of 2 N and moved across the test body surface in a sawtooth pattern. A toothpaste-water mixture serves as abrasion medium (Odol-med 3, 2:1). After 10,000 cycles (simulating a duration of use of approx. 3 to 6 months), the changes on the surface of the composite thus caused are measured and analyzed for roughness (German, Rauigkeit, currently Rauheit, formerly Rauhigkeit, Rauheit: term from the field of surface physics denoting unevenness of the height of a surface) and reflection. Toothbrush abrasion testing apparatus: Manufacturer: Willytec/SD-Mechatronik.

The mastication simulation (CoCoM method, CoCoM method/Computer Controlled Mastication) simulates the abrasion due to an antagonist during a mastication motion. In this context, a ceramic bead made of Al₂O₃ is made to act on the test body surface with a force of 50 N. Once the bead touches the test body surface, the test body is moved sideways by 0.8 mm and the bead abrades the test body surface. Moreover, the test bodies are subjected to a temperature load change before the test (approx. 5,000 cycles between 5° (1 min.)/55° C. (1 min.). A total of 16 test bodies are tested for each material. After 200,000 cycles (simulating a duration of use of approx. 5 years), the damaged caused to the composite is measured and analyzed for depth and volume. Mastication simulation testing apparatus: Manufacturer: Willytec/SD-Mechatronik; the volume in mm³ and the mean depth in μm (micrometers) are measured.

Gloss level: Byk-Gardner gloss measuring device Tri-Gloss, measuring angle 60°

In the following, the advantageous properties according to the invention are evidenced based on examples 1 to 4 according to the invention versus reference examples VG1, VG2, VG3, VG4). The examples summarized in a table containing the same components or compounds were each done using essentially the same amounts of said components or compounds. The components of the compositions are always relative to 100% by weight (total composition).

TABLE 1
Exemplary embodiment: Base mass

| | | VG1 | 1 |
|---|---|---|---|
| Monomers | Bisglycidylacrylate | 3-7 | 3-7 |
| | Urethanedimethacrylate (UDMA)) | 10-15 | 10-15 |
| | alkoxylated pentaerythritol tetraacrylate | 7-10 | 7-10 |
| | TEDMA (triethyleneglycoldimethacrylate) | <3 | <3 |
| Initiators | Butylhydroxytoluene | <1 | <1 |
| | DL-Camphorquinone | <1 | <1 |
| | Benzildimethylketal | <1 | <1 |
| | tert. amine | <0.5 | <0.5 |
| Rheology | Phyllosilicic acid | 14-18 | X |
| modifier | Nano-particulate silicic acid | X | <2 |
| | (trimethylsilyloxy-modified | | |
| | (d50: 2.6 μm) ZrO₂—SiO₂ (d50: 2.6 μm) | X | 20-25 |
| | Gamma-methacryloxypropyl- | <3 | <3 |
| | trimethoxysilane | | |
| Fillers | Glass powder non-sil. | 50-60 | X |
| | (barium-aluminium-borosilicate glass) | | |
| | Glass powder sil. | X | 45-50 |
| | (barium-aluminium-borosilicate glass) | | |

TABLE 2

| Improvement of the abrasion resistance: DA3 | | VG2 | 2 |
|---|---|---|---|
| Monomers | Bisglycidylacrylate | 3-7 | 3-7 |
| | UDMA Urethanedimethacrylate | 10-15 | 10-15 |
| | alkoxylated pentaerythritol tetraacrylate | 7-10 | 7-10 |
| | TEDMA triethyleneglycoldimethacrylate | <3 | <3 |
| Initiators | Butylhydroxytoluene | <1 | <1 |
| | DL-Camphorquinone | <1 | <1 |
| | Benzildimethylketal | <1 | <1 |
| | tert. amine | <1 | <1 |
| Rheology | Phyllosilicic acid | 14-18 | X |
| modifier | Nano-particulate silicic acid | X | <2 |
| | (trimethylsilyloxy-modified) | | |
| | ZrO₂—SiO₂ (d₅₀ 2.6 μm) | X | 20-25 |
| | Gamma-methacryloxypropyl- | <3 | <3 |
| | trimethoxysilane | | |
| Fillers | Glass powder non-sil. | 50-60 | X |
| | (barium-aluminium-borosilicate glass) | | |
| | Glass powder sil. | X | 45-50 |
| | (barium-aluminium-borosilicate glass) | | |
| | Pigments (total) | <1 | <1 |

The use of the filler according to the invention results in marked improvements in toothbrush abrasion (ZB), poppy abrasion (MA) and mastication abrasion (KM).

TABLE 3

| | | | |
|---|---|---|---|
| Abrasion | ZB, depth (μm, micrometer) | 1.29 | 1.04 |
| measurements | ZB, Refl (%) | 2.6 | 4.6 |
| | Gloss level (difference | 87.9 | 39.5 |
| | before-after) | | |
| | MA, depth (μm, micrometer) | 42.6 | 24.1 |
| | MA, Vol (mm³) | 0.4294 | 0.2213 |
| | KM new, depth (μm) | 151.7 | 134.3 |
| | KM new, Vol (mm³) | 0.2108 | 0.1623 |

TABLE 4

| | VG3 | 3 | 4 | VG4 |
|---|---|---|---|---|
| Bisglycidylacrylate | 3-7 | 3-7 | 3-7 | 3-7 |
| UDMA Urethanedimethacrylate | 10-15 | 10-15 | 10-15 | 10-15 |
| alkoxylated pentaerythritol tetraacrylate | 6-10 | 6-10 | 6-10 | 6-10 |
| TEDMA | <3 | <3 | <3 | <3 |
| DL-Camphorquinone | <1 | <1 | <1 | <1 |
| Butylhydroxytoluene | <1 | <1 | <1 | <1 |
| Benzildimethylketal | <1 | <1 | <1 | <1 |
| tert. amine | <1 | <1 | <1 | <1 |
| ZrO₂—SiO₂ (d₅₀ 2.6 μm) | | 18-25 | 11-15 | |
| Merck JE340 4-8 μm (micrometer) | | | | 14 |
| Gamma-methacryloxypropyl- | <3 | <3 | <3 | |
| trimethoxysilane | | | | |
| Nano-particulate silicic acid | | | <2 | <2 |
| (trimethylsilyloxy-modified) | | | | |
| Phyllosilicic acid | 14-18 | | | |
| Glass powder sil. | 50-65 | 50-65 | 50-65 | 50-65 |
| (barium-aluminium-borosilicate glass) | | | | |
| % by weight | 100 | 100 | 100 | 100 |

TABLE 5

| | | VG3 | 3 | 4 | VG4 |
|---|---|---|---|---|---|
| 3-point flexural test [MPa] | | 141.2 | 142.4 | 139.1 | 147 |
| (HiLite Power 180/90 sec. both sides) | | 10224 | 10919 | 9024 | 10720.0 |
| Color values | L (brightness) | 91.12 | 91.43 | 91.58 | 87.21 |
| (8 min Palatray | a (red/green) | −2.75 | −1.01 | −1.43 | −1.88 |
| CU - illumination | b (yellow/blue) | 13.73 | 6.22 | 6.17 | 10.13 |
| on both sides.) | C (Chroma) | 14 | 6.3 | 6.34 | 10.3 |
| (HiLite Power 180 sec. | h (hue) | 101.32 | 99.19 | 103.03 | 100.49 |
| both sides) | T (Transparency) | 57.2 | 64.15 | 59.44 | 41.16 |
| | [%] | | | | |

| | | | | |
|---|---|---|---|---|
| Toothbrush Reflection [%] | 4.70 | 7.90 | 7.60 | 7.30 |
| Toothbrush Roughness [μm] | 0.5200 | 0.38 | 0.49 | 0.54 |
| MA depth (μm) | 39.80 | | 20.9 | |
| MAVol. (mm³) | 0.3886 | | 0.1753 | |

Gloss level: Byk-Gardner gloss measuring device Tri-Gloss, measuring angle 60°

The use of the filler according to the invention results in marked improvements in the transparency of the non-dyed base mass. Silicon dioxide- and zirconium dioxide-containing (ZrO₂—SiO₂) products from other manufacturers do not show these positive effects.

1. Dental material comprising (a) at least one curable monomer and/or polymer component, and (b) at least one filler component the at least one filler component (b) comprises agglomerated oxide particles, wherein the oxide particles comprise a matrix and a doping component, whereby the matrix comprises silicon dioxide and the doping component comprises zirconium dioxide.

2. Dental material according to claim 1, wherein the agglomerated oxide particles comprise agglomerates of silicon dioxide primary particles doped with zirconium dioxide, whereby the agglomerates have a particle size of 0.1 μm or more to 12 μm or less.

3. Dental material according to claim 1, wherein the oxide particles are silicon dioxide-containing primary particles comprising micro-crystalline domains of 4 to 7 nm, and optionally the oxide particles comprise micro-crystalline zirconium dioxide-containing domains.

4. Dental material according to claim 1, wherein the agglomerated oxide particles comprising the matrix and the doping component comprise a mixture of metal dioxides selected from silicon dioxide and zirconium dioxide.

5. Dental material according to claim 1, wherein the agglomerated oxide particles contain 1 to 25% by weight zirconium dioxide, relative to their total composition.

6. Dental material according to claim 1, wherein the agglomerated oxide particles comprise 75 to 80% by weight silicon dioxide and 20 to 25% by weight zirconium dioxide, relative to their total composition.

7. Dental material according to claim 1, wherein the agglomerated oxide particles comprise silicon dioxide and zirconium dioxide at a molar ratio of 1 to 9.

8. Dental material according to claim 1, wherein the refractive index of the agglomerated oxide particles is between 1.49 and 1.55.

9. Dental material according to claim 1 wherein the filler component comprises agglomerated oxide particles, which comprise agglomerates of primary particles made up of silicon dioxide and zirconium dioxide, whereby the agglomerates have a particle size d₅₀ of 0.5 to 10 μm.

10. Dental material according to claim 1, wherein
the agglomerated oxide particles have a surface modification through surface occupation by an organo-functional silane.

11. Dental material according to claim 1, wherein the agglomerated oxide particles are surface-modified by conversion products of methacryloxyalkylenetrialkoxysilane, 3-methacryloxypropyltrimethoxysilane and/or 3-methacryloxypropyltriethoxysilane.

12. Dental material according to claim 1, wherein the agglomerated oxide particles have a grain size distribution d₉₀ of 12 μm or less and a mean particle size d₅₀ of approx. 2.4 to 3.0 μm.

13. Dental material according to claim 1, wherein the agglomerated oxide particles are present at up to 80% by weight, relative to the total composition of the dental material.

14. Dental material according to claim 1, wherein the dental material comprises:
(a) at least one curable monomer and/or polymer component,
(b) at least one filler component comprising (b.1) at least agglomerated oxide particles having a matrix and a doping component, whereby the matrix comprises silicon dioxide and the doping component comprises zirconium dioxide, (b.2) glasses and/or mixtures of at least two of the afore-mentioned filler components, and, optionally, (c) at least one initiator.

15. Dental material according to claim 1, wherein the filler component comprising agglomerated oxide particles comprises agglomerates of primary particles of silicon dioxide and zirconium dioxide, whereby the primary particles have an average particle diameter of at least approx. 3 to 70 nm.

16. Dental material according to claim 1, wherein (a) the agglomerated oxide particles, (b) the agglomerated and aggregated oxide particles and/or (c) the surface-modified agglomerated oxide particles, (a), (b) or (c) in each case, are not cleaved into the primary particles by high shear forces that occur during the production of dental materials.

17. Dental material according to claim 1, wherein the agglomerated oxide particles have an index of crystallinity of 0.6 to 0.7.

18. Dental material according to claim 1, wherein the dental material is a filling composite, veneer composite, green compact of an artificial tooth, green compact of a veneer, green compact of an inlay, green compact of an implant, green compact of a carrier material for a local release of pharmaceutically active substances, green compact of a carrier material for a local antibiotics therapy or a green compact of a cutting block for the manufacture of dental prostheses according to the CAD/CAM technique or at least a part of the afore-mentioned dental materials.

19. Method for producing a dental material according to claim 1, said method comprising mixing (a) at least one curable monomer and/or polymer component, and
(b) at least one filler component comprising
(b.1) at least agglomerated oxide particles having a matrix and a doping component, whereby the matrix comprises silicon dioxide and the doping component comprises zirconium dioxide.

20. Method according to claim 19, which further comprises, in addition, mixingas filler component (b), (b.2) glasses and/or mixtures of at least two of the afore-mentioned filler components and, optionally, (c) an initiator.

21. Method according to claim 19, wherein the dental material (1) is formed and (2) optionally polymerized.

22. Cured dental material obtainable by mixing, optionally forming and polymerizing, a dental material according to claim 1.

23. Dental material according to claim 1 obtainable by polymerization and that has a transparency in excess of 58 (illumination: 8 min Palatray CU, both sides, HiLite Power 180 sec. both sides), a gloss level difference of 45 or less after toothbrush abrasion (polished surface up to 1000/2500/4000-grit polishing paper, diamond suspension yellow/red/white, toothbrush abrasion simulation Willytec/SD-Mechatronik, toothbrush Hager & Werken, Odol-med-3, 10,000 cycles, sawtooth profile, equivalent to approx. 3-6 months of brushing), a roughness of less than 30 μm depth after poppy abrasion, a roughness of 0.3500 mm³ volume or less and/or with a reflection in excess of 2.5% as determined after a toothbrush abrasion or a dental material comprising at least two of the afore-mentioned parameters.

24. Dental material according to claim 1 obtainable by polymerization, wherein the agglomerated and, optionally, aggregated oxide particles are incorporated into a polymer matrix of the dental material and are removed layer-by-layer together with the polymer matrix of the dental material during an abrasion process, rather than chipping away as individual intact particles.

25. Dental material according to claim 22, wherein the dental material is an artificial tooth, veneer, inlay, implant, carrier material for a local release of pharmaceutically active substances, carrier material for a local antibiotics therapy or a cutting block for the manufacture of dental prostheses according to the CAD/CAM technique or at least a part of the afore-mentioned dental materials.

26. Filler component comprising agglomerated oxide particles having a matrix and a doping component, whereby the matrix contains silicon dioxide and the doping component comprises zirconium dioxide, and whereby the agglomerated oxide particles are surface-modified by an organo-functional silane.

27. Filler component according to claim 26, wherein the agglomerated oxide particles are surface-modified by conversion products of olefinic alkoxysilanes, in particular of 3-methacryloxytrimethoxysilane and/or 3-methacryloxytriethoxysilane.

28. Method of using a dental material according to claim 1 for producing a filling composite, veneer composite, veneer, artificial tooth, inlays, cements, dental prostheses, for producing carrier materials for a local antibiotics therapy or as carrier material for a local release of pharmaceutically active substances, cutting blocks for producing dental prostheses according to the CAD/CAM technique.
